(19) United States
(12) Patent Application Publication
(10) Pub. No.: US 2019/0298191 A1
(43) Pub. Date: Oct. 3, 2019

(54) ADAPTIVE TRANSFER FUNCTION FOR DETERMINING CENTRAL BLOOD PRESSURE

(71) Applicant: Board of Trustees of Michigan State University, East Lansing, MI (US)

(72) Inventors: Ramakrishna MUKKAMALA, Okemos, MI (US); Mingwu GAO, Boston, MA (US)

(73) Assignee: Board of Trustees of Michigan State University, East Lansing, MI (US)

(21) Appl. No.: 16/080,787

(22) PCT Filed: Apr. 19, 2017
(86) PCT No.: PCT/US17/28314

§ 371 Date: Aug. 29, 2018

Related U.S. Application Data
(60) Provisional application No. 62/324,493, filed on Apr. 19, 2016.

(51) Int. Cl. A61B 5/021; A61B 5/0215; A61B 5/022; A61B 5/00

(52) U.S. Cl. CPC A61B 5/02125; A61B 5/0215; A61B 5/02241; A61B 5/7278; A61B 5/02216; A61B 5/7221; A61B 5/725; A61B 5/02225

(57) ABSTRACT

Generalized transfer functions are available to mathematically derive the more relevant central blood pressure waveform from a more easily measured radial blood pressure waveform. However, these transfer functions are population averages and therefore may not adapt well to variations in pulse pressure amplification (ratio of radial to central pulse pressure). An adaptive transfer function was developed. First, the transfer function is represented in terms of the wave travel time and wave reflection coefficient parameters of an arterial model. Then, the model parameters are estimated from only the radial blood pressure waveform by exploiting the frequent observation that central blood pressure waveforms exhibit exponential diastolic decays. The adaptive transfer function estimated central blood pressure with significantly greater accuracy than generalized transfer functions in the low pulse pressure amplification group while showing similar accuracy to the conventional transfer functions in the higher pulse pressure amplification groups.

### CROSS-REFERENCE TO RELATED APPLICATIONS

This application is a 371 National Phase of International Application PCT/US2017/028314, filed Apr. 19, 2017, which claims the benefit of U.S. Provisional Application No. 62/324,493, filed on Apr. 19, 2016. The entire disclosure of the above applications are incorporated herein by reference.

### GOVERNMENT CLAUSE

This invention was made with government support under AG041361 awarded by the National Institutes of Health, and under U.S. Pat. No. 1,403,004 awarded by the National Science Foundation. The government has certain rights in the invention.

### FIELD

The present disclosure relates to an adaptive method for determining central blood pressure from a peripheral blood pressure measure.

### BACKGROUND

Blood pressure (BP) waveforms become progressively distorted with increasing distance from the heart. Most notably, pulse pressure (PP) becomes increasingly amplified. This counter-intuitive phenomenon is mainly caused by wave transmission and reflection in the arterial tree. The extent of the amplification can vary with, for example, BP- and age-induced changes in the wave travel time (which indicates the speed of the wave) and peripheral resistance-induced changes in the wave reflection coefficient (which indicates the relative magnitude of the reflected wave). So, it is BP near the heart (i.e., central BP) that directly reflects and affects cardiac performance. Further, central BP, rather than BP away from the heart (i.e., peripheral BP), is a major determinant of the degenerative changes that occur in aging and hypertension. Because of its greater physiologic relevance, central BP could provide superior clinical value. However, peripheral BP waveforms are easier to measure via catheterization and applanation tonometry of a radial artery (at the wrist).

O'Rourke and co-workers previously proposed to mathematically derive the central BP waveform from a radial BP waveform. They developed an average transfer function (i.e., a frequency-dependent transformation) to relate measured radial BP waveforms to measured central BP waveforms from a group of subjects and then applied the transfer function to the radial BP waveform of new subjects to predict the central BP waveform. Thereafter, others showed that this “generalized transfer function” (GTF) could yield good agreement with invasive central BP measurements in cardiac catheterization patients. These initial, independent validation studies have received considerable attention and helped popularize the GTF.

However, since the GTF is a population average, it could often effectively assume that the PP amplification (the ratio of radial PP to central PP) is simply a fixed value. Hence, the GTF may not adapt to the aforesaid inter-subject and temporal variability in PP amplification and therefore yield nontrivial central BP errors when the PP amplification is atypical. An improved transfer function could help enhance the clinical utility of central BP, which has only been able to demonstrate marginal added clinical value over peripheral BP up to now.

This section provides background information related to the present disclosure which is not necessarily prior art.

### SUMMARY

This section provides a general summary of the disclosure, and is not a comprehensive disclosure of its full scope or all of its features.

A method is provided for determining central blood pressure for a subject. The method includes: measuring, by a sensor, a peripheral blood pressure waveform from the subject; defining a model that relates the measured peripheral blood pressure waveform to a central blood pressure waveform, where the model is defined in terms of parameters representing wave travel time and wave reflection coefficient; and determining central blood pressure for the subject by selecting the parameters and applying the model to the measured peripheral blood pressure in a manner that yields smallest error in fitting of an exponential function to a diastolic interval of the central blood pressure.

In one embodiment, the method further includes: measuring, by a sensor, a peripheral blood pressure waveform from the subject; and defining a model that relates the measured peripheral blood pressure waveform to a central blood pressure waveform, where the model is defined in terms of parameters reflecting wave travel time and wave reflection coefficient. Multiple sets of candidate values for wave travel time and wave reflection coefficient parameters are selected. For each set of candidate values, a candidate central blood pressure waveform is computed by applying the model with a given set of candidate values to the measured peripheral blood pressure waveform. For each candidate central blood pressure waveform, an exponential is then fitted to the diastolic decay of a given candidate central blood pressure waveform. Prior to the step of fitting, each candidate central blood pressure waveform may be low pass filtered. Lastly, the central blood pressure for the subject is determined to be the candidate central blood pressure waveform having smallest fitting error between the exponential and the diastolic decay.

The peripheral blood pressure waveform may be measured using a catheter or a finger-cuff photoplethysmograph or an applanation tonometer or an oscillometric cuff.

The model may be further defined as a tube-load model, where the tube represents wave travel path between central aorta and a peripheral artery and terminal loads represent the arterial bed distal to the peripheral artery. More specifically, the method is defined as

$${P}_{c} ( t ) = \frac{1}{1 + Γ} {P}_{r} ( t + {T}_{d} ) + \frac{Γ}{1 + Γ} {P}_{r} ( t - {T}_{d} )$$

where P_{c}(t) is the central blood pressure waveform, Pᵣ(t) is the measured peripheral blood pressure waveform, T_{d} is the wave travel time and Γ is the wave reflection coefficient.

In some embodiments, multiple sets of candidate values for wave travel time and wave reflection coefficient are selected from respective physiological ranges of values.

In other embodiments, fitting an exponential further comprises estimating a diastolic interval of the given candidate central blood pressure waveform using pulse length; and applying a logarithm operation to the estimated diastolic interval of the given candidate central blood pressure waveform and fitting a line to the log transformed data.

In another aspect, a variant method is provided for determining central blood pressure for a subject. The method includes: measuring, by a sensor, a peripheral blood pressure waveform from the subject; defining a model that relates the measured peripheral blood pressure waveform to a central blood pressure waveform, where the model is defined in terms of parameters representing the wave travel time and wave reflection coefficient; determining the parameters representing the wave reflection coefficient based on population averages; determining the parameters representing wave travel time based on its inverse relationship with blood pressure; and determining central blood pressure by applying the determined model to the measured peripheral blood pressure waveform, where the step of determining is executed by a computer processor of a computing device.

In yet another aspect, a computer-implemented system is provided for determining central blood pressure for a subject. The system includes: a sensor configured to measure a peripheral blood pressure waveform of the subject; a data store that stores a model that relates the measured peripheral blood pressure waveform to a central blood pressure waveform, where the model is defined in terms of wave travel time and wave reflection coefficient; and a model estimation module configured to receive the measured peripheral blood pressure waveform from the sensor and to receive multiple sets of candidate values for wave travel time and wave reflection coefficient. For each set of candidate values, the model estimation module computes a candidate central blood pressure waveform by applying the model with a given set of candidate values to the measured peripheral blood pressure waveform and fits, for each candidate central blood pressure waveform, an exponential to diastolic decay of a given candidate central blood pressure waveform. The model estimation module is implemented by computer readable instructions executed by a computer processor residing on a computing device.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a flowchart depicting an improved method for determining central blood pressure for a subject;

FIG. 2 is diagram depicting an example arterial model that relates a measured peripheral blood pressure waveform to a central blood pressure waveform;

FIG. 3 is a diagram illustrating an example technique for estimating the model parameters by making the waveform exhibit maximally exponential diastolic decays;

FIGS. 4A-4D are graphs depicting the estimated (dashed) and measured (dark) central blood pressure waveforms for an autoregressive exogenous input-based generalized transfer function (GTF_{ARX}), a generalized transfer function that mimics the SphygmoCor device of AtCor Medical (GTF_{SphygmoCor}) and the proposed adaptive method (ATF), respectively, in the case of a low ratio of radial to central pulse pressure;

FIGS. 4E-4H are graphs depicting the estimated (dashed) and measured (dark) central blood pressure waveforms for an autoregressive exogenous input-based generalized transfer function (GTF_{ARX}), a generalized transfer function that mimics the SphygmoCor device (GTF_{SphygmoCor}) and the proposed adaptive method (ATF), respectively, in the case of a middle ratio of radial to central pulse pressure;

FIGS. 41-4L are graphs depicting the estimated (dashed) and measured (dark) central blood pressure waveforms for an autoregressive exogenous input-based generalized transfer function (GTF_{ARX}), a generalized transfer function that mimics the SphygmoCor device (GTF_{SphygmoCor}) and the proposed adaptive method (ATF), respectively, in the case of a high ratio of radial to central pulse pressure;

FIGS. 5A and 5B are graphs depicting average wave travel time T_{d} and wave reflection coefficient Γ (mean±SE) parameter estimates, respectively, of the proposed adaptive method; and

FIG. 6 is a block diagram of an apparatus for implementing the methods according this disclosure.

Corresponding reference numerals indicate corresponding parts throughout the several views of the drawings.

### DETAILED DESCRIPTION

Example embodiments will now be described more fully with reference to the accompanying drawings.

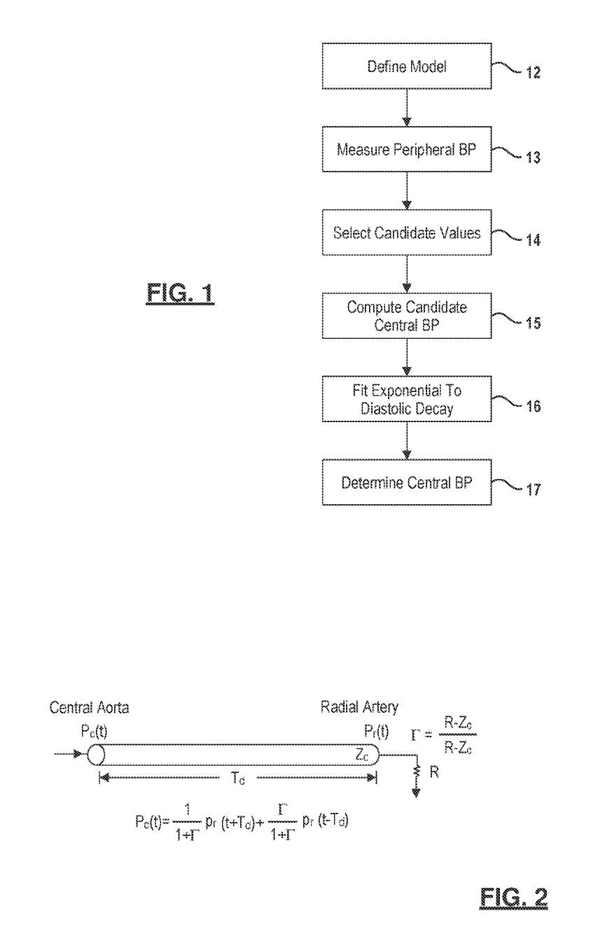

With reference to FIG. 1, an improved method is provided for determining central BP in a subject from a measured peripheral BPwaveform. First, a model (e.g., transfer function) that relates a measured peripheral BP waveform to a central BP waveform is defined at 12 in terms of an arterial model with unknown parameters. In the example embodiment, the model is an arterial tube-load model, and the unknown parameters are wave travel time and wave reflection coefficient. Although other models and parameters are also contemplated (e.g., model parameters reflecting a frequency-dependent wave reflection coefficient), the model is further described below.

Next, a peripheral BP waveform of the subject is measured at 13 by a sensor at 13. In some embodiments, the sensor may be an invasive catheter (in a radial or other artery), a finger-cuff photoplethysmograph (operating the volume clamp method), an applanation tonometer, or an oscillometric arm cuff (operating at a standard varying cuff pressure to determine systolic and diastolic BP followed possibly by a fixed cuff pressure to obtain a pulse volume plethysmography waveform that is then calibrated to the systolic and diastolic BP levels). Other types of invasive and non-invasive sensors are also contemplated by this disclosure.

Parameter values for the model are estimated by exploiting the observation that central BP exhibits exponential diastolic decays. Multiple sets of candidate values are selected at 14 for wave travel time and wave reflection coefficient. For each set of candidate values, a candidate central BP waveform is computed at 15 by applying the model with a given set of candidate values to the measured peripheral BP waveform, and an exponential is then fitted at 16 to the diastolic decay of the computed candidate central BP waveform. Lastly, central BP is deemed to be the candidate central BP waveform having the smallest fitting error between the exponential and the diastolic decay and is selected as indicated at 17. Each of these steps is further described below.

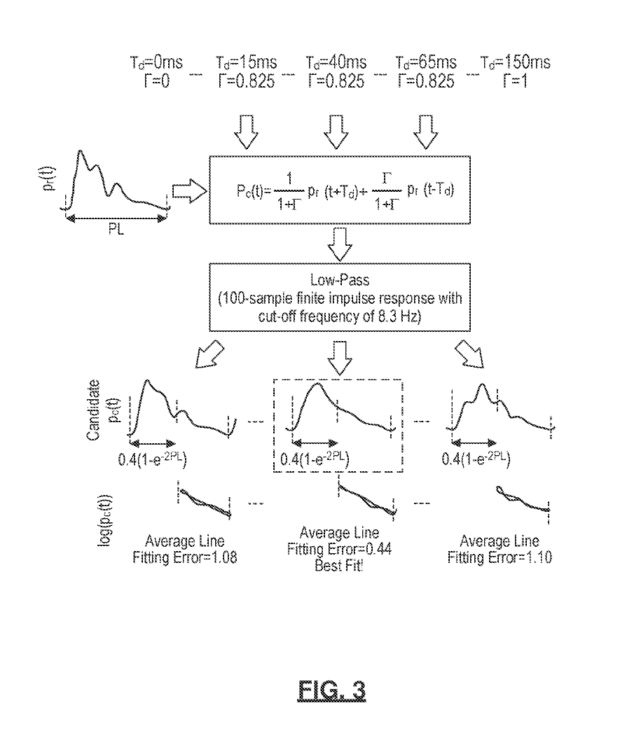

FIG. 2 depicts an example model that relates a measured peripheral BP waveform to a central BP waveform. More specifically, a tube-load model is employed to represent arterial wave transmission and reflection. The tube represents the wave travel path between the ascending aorta and a radial artery, while the terminal load represents the arterial bed distal to the radial artery. Note that the wave travel path to other peripheral arteries could be represented by placing similar combinations of tubes and loads in parallel. The tube accounts for arterial inertance [L] and compliance [C] and therefore exhibits constant characteristic impedance [Zc=√(L/C)] and allows waves to travel along the entire tube with constant time delay or wave travel time [Td=√(LC)]. The load accounts for the peripheral resistance [R]. While previous tube-load models have represented the load with a more complicated, three-parameter Windkessel model, the purely resistive load may often suffice. Waves traveling in the forward direction (left-to-right) along the tube are reflected in the backward direction (right-to-left) at the terminal load with a constant reflection coefficient (Γ=(R−Zc)/(R+Zc)) so as to mimic the progressive amplification that BP waveforms undergo with increasing distance from the heart. According to this model, the transfer function relating radial BP [Pr(t)] (i.e., BP at the tube end) to central BP [Pc(t)] (i.e., BP at the tube entrance) may be defined in terms of two parameters, wave travel time Td and wave reflection coefficient Γ.

In an example embodiment, the two model parameters values, and thus the central BP waveform, are estimated from only the radial BP waveform, for example sampled at 200 Hz. First, multiple sets of candidate values are selected for the wave travel time and the wave reflection coefficient from respective physiological ranges of values. For example, values for wave travel time Td are selected from the wide range of 0 to 150 ms, in increments of 5 ms; whereas, values for wave reflection coefficient are selected in the physical range of 0 to 1, in increments of 0.05. It is understood that values may be selected at different and/or varying increments.

Second, a candidate central BP waveform is computed by applying the time-domain model equation, equipped with the two selected parameter values, to the radial BP waveform. That is, a candidate central BP waveform is computed for each set of candidate values in the multiple sets of candidate values selected above. In the example embodiment, the model (or transfer function) is as follows:

$${P}_{c} ( t ) = \frac{1}{1 + Γ} {P}_{r} ( t + {T}_{d} ) + \frac{Γ}{1 + Γ} {P}_{r} ( t - {T}_{d} )$$

where P_{c}(t) is the central BP waveform, Pᵣ(t) is the measured peripheral BP waveform, T_{d} is the wave travel time and Γ is the wave reflection coefficient.

Third, for each candidate central BP waveform, an exponential is fitted to the diastolic decay of a given candidate central BP waveform. In one embodiment, the diastolic interval is estimated in the given candidate central BP waveform using the preceding pulse length. For example, the diastolic interval (DI) of each beat of the candidate central BP waveform is approximated from the preceding pulse length (PL) according to the following formula: DI=PL−0.4(1−e^{-2-PL}). Other techniques for estimating the diastolic interval also fall within the broader aspects of this disclosure. An exponential is then fitted to the estimated diastolic decay interval of the given candidate central BP waveform. That is, the candidate central BP over each DI is log transformed, and a line is fitted to this data using standard linear regression.

Prior to the step of fitting, each candidate central BP waveform can optionally be low pass filtered. For example, a 100-sample finite impulse response low-pass filter may be applied to further smooth the candidate waveform with a cutoff frequency between 5 to 10 Hz.

Lastly, a central BP is determined for the subject. In the example embodiment, the central BP is deemed to be the candidate central BP waveform having smallest fitting error between the exponential and the diastolic decay. The fitting error may be the average square fitting error over all of the beats. In the example embodiment, the above steps are repeated for every pair of candidate values, Td and Γ, to arrive at a set of candidate central BP waveforms. The Td and Γ values and candidate central BP waveform that yield the minimum fitting error are chosen as the final estimates for central BP.

In an alternative embodiment, the wave travel time and the wave reflection coefficient are estimated in a different manner. Since the transformation is relatively insensitive to the wave reflection coefficient (see below) and since wave travel time may be reasonably predicted from available data, a basic regression approach is applied to determine the parameter values per subject. Based on a training dataset, the wave reflection coefficient is set to a constant, for example a population average. The wave travel time is predicted from only the mean (or another parameter such as the minimum) of the peripheral BP waveform, which is well known to be a strong predictor of this parameter, via a line with non-zero intercept. Alternatively, the wave travel time could also be predicted from additional parameters such as age and height. Once the parameters are known, then the central BP waveform can be computed from the equation provided above.

Further, in some embodiments, other physiologic parameters may be derived from the estimated central BP waveform and the wave travel time and wave reflection coefficient parameters. For example, cardiac output may be computed to within a scale factor. In one such embodiment, a mean BP level divided by the time constant of the best exponential fit may be determined. In another embodiment, central PP times the pulse rate may be determined. In yet another embodiment, the following equation may be used to compute the central blood flow rate waveform (qc):

$${Z}_{c} {q}_{c} ( t ) = \frac{1}{1 + Γ} {P}_{r} ( t + {T}_{d} ) - \frac{Γ}{1 + Γ} {P}_{r} ( t - {T}_{d} )$$

This waveform is then averaged to derive cardiac output to within a scale factor. In all cases, as is common in practice, the cardiac output may then possibly be corrected for changes in arterial compliance (and inertance) based on the measured BP levels and subject anthropomorphic information (e.g., age, height, weight, gender) via a nomogram. As another example, left ventricular ejection fraction may be computed from the estimated central BP waveform using a ventriculo-arterial model. The ejection fraction may be periodically calibrated with an imaging measurement to determine its unstressed volume component if desired. Further information may be found in U.S. Pat. No. 8,282,569 which is incorporated herein in its entirety.

The adaptive transfer function (ATF) method described above was assessed and compared to GTFs using patient data that was previously collected under institutional review board approval from the Johns Hopkins Hospital and originally used for initial, independent validation of the GTF. Briefly, the data were from two cohorts of cardiac catheterization patients. The first cohort comprised 20 patients with a hemodynamic intervention to transiently change BP in 14 of the subjects. The second cohort consisted of 19 patients without any intervention. Each patient record included a radial BP waveform via an applanation tonometer and the reference central BP waveform via a micromanometer-tipped ascending aortic catheter. Both waveforms were 10-35 sec in duration, sampled at 200 Hz, and low-pass filtered with a cutoff frequency of 15 Hz. Three of the interventions produced changes in central BP levels that lasted less than 10 beats. Since the ATF and perhaps even the GTF require steady periods of data for their construction, the post-intervention waveforms for the corresponding patient records were excluded from subsequent data analysis. Table 1 summarizes the patient and data characteristics.

TABLE 1
Patient and data characteristics

| Patient Characteristics | Cohort 1 (n = 20) | Cohort 2 (n = 19) |
|---|---|---|
| Men [%] | 80 | 74 |
| Age [years] | 59 ± 11 | 51 ± 16 |
| Post Heart Transplant [%] | 50 | 26 |
| Coronary Artery Disease [%] | 10 | 58 |
| Dilated Cardiomyopathy [%] | 35 | 0 |
| Constrictive Pericarditis [%] | 5 | 0 |
| Normal [%] | 0 | 11 |
| Hypertension [%] | 0 | 5 |
| Data Characteristics (Baseline) | Cohort 1 (n = 20) | Cohort 2 (n = 19) |
| Central PP [mmHg] | 59 ± 15 | 48 ± 17 |
| Radial PP [mmHg] | 69 ± 28 | 52 ± 11 |
| DP [mmHg] | 86 ± 15 | 79 ± 18 |
| Data Characteristics (Intervention) | Cohort 1 (n = 11) | Cohort 2 (n = 0) |
| Valsalva Maneuver [%] | 55 | — |
| Nitroglycerin [%] | 9 | — |
| Abdominal Compression [%] | 27 | — |
| Inferior Vena Cava [%] | 9 | — |
| \|Central PP Change\| [mmHg] | 16 ± 11 | — |
| \|Radial PP Change\| [mmHg] | 14 ± 12 | — |
| \|DP Change\| [mmHg] | 24 ± 15 | — |

Similar to the original, independent validation studies of the GTF, the radial BP waveforms were calibrated to the mean and diastolic levels of the reference central BP waveforms in order to focus on the transfer function itself in absence of the confounding effect of the BP calibration. The patient records in the first cohort were used to train the ATF and GTFs, while the patient records in the second cohort were used to test the transfer functions. The roles of the first and second cohorts were then interchanged, and the training and testing procedure was repeated. In this way, the patient records in both cohorts were utilized to assess the transfer functions without employing the same data for training and testing.

The ATF was trained in terms of the cutoff frequency of the post-low-pass filter and the type of load (resistor versus three-parameter Windkessel). For comparison, three GTFs were also trained. The first GTF was constructed based on the autoregressive exogenous input (ARX) identification procedure outlined in the original, independent validation study. This procedure was shown to be most effective amongst various approaches in that study. The second GTF was constructed based on a more straightforward ARX identification procedure. In particular, one half of each pair of radial and central BP waveforms was utilized to determine the time delay ranging from ˜30 to 0 samples and the ARX parameters for model orders ranging from 1 to 15 using standard least squares estimation. The other half of each pair of waveforms was then employed to determine which of the 15 ARX-based transfer functions yielded the minimum average square central BP waveform estimation error. The optimal transfer functions from each pair of waveforms were then averaged to arrive at the final GTF. This second GTF (GTF_{ARX}) estimated central BP more accurately than the first GTF in the testing data, and varying its model order range did not further improve the estimation (results not shown). The third GTF was built by reverse engineering the SphygmoCor device (AtCor Medical, Australia). This GTF (GTF_{SphygmoCor}) was a 34-sample finite impulse response filter at a sampling frequency of 128 Hz that was virtually identical to the device transfer function (results not shown). The GTF_{SphygmoCor} was therefore investigated after resampling the waveforms to 128 Hz.

The testing data was divided into low, middle, and high PP amplification groups of equal sizes, and the following analysis was applied to each group. The central BP waveforms estimated by the ATF, GTF_{ARX}, and GTF_{SphygmoCor} were quantitatively evaluated against the reference central BP waveforms in terms of the sample-to-sample (total waveform, TW), average systolic BP (SP), and average PP root-mean-squared-errors (RMSEs). The analyzed radial BP waveforms were likewise evaluated. All waveforms were time aligned with the reference waveforms prior to the TW RMSE calculation. The RMSEs for the ATF were then statistically compared to the RMSEs for the two GTFs and the radial BP waveform via paired t-tests of the squared-errors with Holm's correction for the three comparisons. In addition, the T_{d} and Γ estimates of the ATF were statistically compared between pairs of the three PP amplification groups via two-sample t-tests again with Holm's correction for the three comparisons.

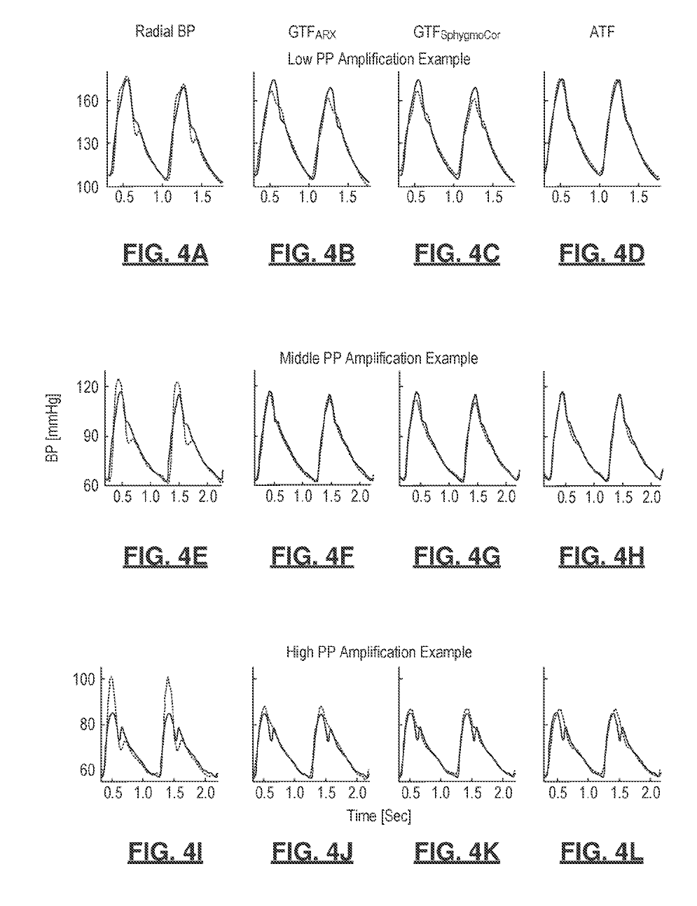

The ATF implemented with a purely resistive load performed essentially the same as the ATF implemented with a conventional three-parameter Windkessel load in the training data. Hence, in the example embodiment, the simpler load was selected. In other embodiments, the three-parameter Windkessel load or other loads may be used. The post-low-pass filter cutoff frequency for the ATF was 8.4 Hz when the first cohort of patient records was used as the training data and 7.9 Hz when the second cohort was used as the training data. Hence, despite the use of two training datasets, the ATF could be represented with a single procedure, as shown in FIG. 3. Note that a post-low-pass filter did not improve the central BP estimates of the GTFs.

TABLE 2
Root-mean-squared-errors between estimated
and measured central blood pressure (BP)

| | Low PP | | | Middle PP | | | High PP | | |
|---|---|---|---|---|---|---|---|---|---|
| | Amplification | | | Amplification | | | Amplification | | |
| Central BP | (1.06 ± 0.07) | | | (1.25 ± 0.07) | | | (1.59 ± 0.13) | | |
| Estimates | TW | SP | PP | TW | SP | PP | TW | SP | PP |
| Radial BP | 6.6* | 6.1# | 6.1 | 7.8* | 13.9* | 13.9* | 8.1* | 21.6* | 21.6* |
| GTF_{SphygmoCor} | 4.7* | 7.5* | 10.1* | 3.5 | 5.4 | 7.9* | 2.9 | 3.1 | 4.8 |
| GTF_{ARX} | 5.2* | 6.2# | 7.1* | 3.2 | 3.5 | 4.6 | 2.9 | 3.5 | 4.3 |
| ATF | 3.5 | 3.3 | 4.2 | 3.5 | 3.3 | 3.4 | 3.1 | 3.7 | 3.7 |

* and # denote statistically different (e.g., p < 0.05) or borderline statistically different (e.g., p ≈ 0.05) compared to ATF, respectively.

Table 2 shows the central TW, SP, and PP RMSEs for the radial BP waveform, GTF_{SphygmoCor}, GTF_{ARX}, and ATF in the testing data for the low, middle, and high PP amplification groups. The average (mean±SD) PP amplification was 1.06±0.07 for the low group, 1.25±0.07 for the middle group, and 1.59±0.13 for the high group.

As expected, the RMSEs for the radial BP waveform were very large but decreased substantially with PP amplification. The RMSEs for the GTF_{SphygmoCor} were lowest in the high PP amplification group rather than the middle PP amplification group and were highest in the low PP amplification group. Also as expected, the RMSEs for the GTF_{ARX} were low in the middle PP amplification group and higher in the low PP amplification group. However, this transfer function surprisingly yielded low RMSEs for the high PP amplification group. By contrast, the RMSEs for the ATF were comparable in all three PP amplification groups. Further, the RMSEs for the ATF were considerably lower than those for the radial BP waveform in all three groups, significantly lower than those for both GTFs in the low PP amplification group, and even lower than those for the GTF_{SphygmoCor} in the middle PP amplification group. Most notably, in the low PP amplification group, the ATF showed average RMSE reductions of 40% relative to the GTF_{ARX} and nearly 50% relative to the GTF_{SphygmoCor}.

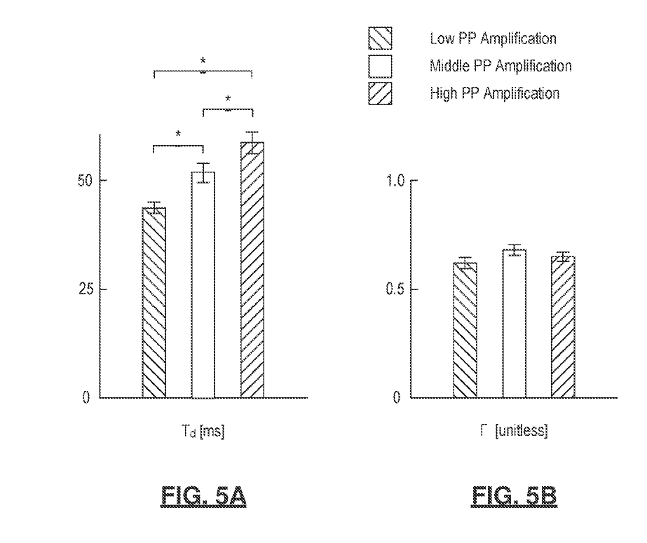

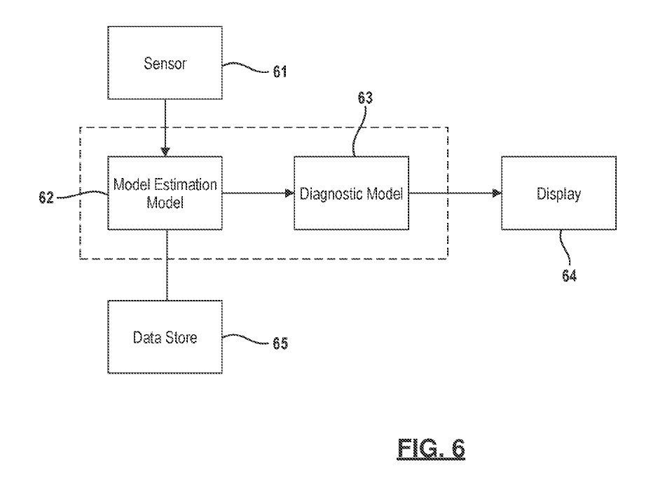

FIGS. 4A-4L depicts representative examples of the estimated and measured central BP waveforms in the testing data for the low, middle, and high PP amplification groups. As can be seen, the ATF provided the best central BP waveform estimates over all three examples.

FIGS. 5A and 5B shows the average T_{d} and Γ estimates of the ATF in the testing data for the low, middle, and high PP amplification groups, respectively. The T_{d} estimates significantly increased with PP amplification, whereas the Γ estimates did not change. Since PP amplification can increase with T_{d}, Γ, or T_{d} and Γ, these parameter estimates give further credence to the ATF.

Thus, a simple adaptive transfer function (ATF) was developed for mathematically deriving the central BP waveform from a radial BP waveform. The transfer function is defined in terms of wave travel time and wave reflection coefficient parameters of a physical model of arterial wave transmission and reflection (see FIG. 2). The model parameters are then estimated from only the radial BP waveform by assuming that the central BP waveform exhibits exponential diastolic decays (see FIG. 3). In this way, unlike conventional GTFs, the transfer function may effectively adapt to the arterial properties of the subject at the time of measurement.

Frank first proposed that central BP waveforms could be represented with a Windkessel model, which predicts exponential diastolic decays. Thereafter, exponential diastolic decays in the central BP waveform have been repeatedly observed. The mechanism for such diastolic decays may be as follows. Forward and backward waves in the aorta have large phasic differences due to the long and varying distances between the aorta and the main reflection sites at the arterial terminations. Hence, waves with short wavelengths tend to cancel each other out in the aorta. On the other hand, waves with longer wavelengths build up in the aorta. However, these wavelengths may be long relative to the dimension of the arterial tree such that it indeed acts like a Windkessel from the perspective of the aorta. The physical model upon which the ATF is based in FIG. 2 captures this mechanism to a significant, but incomplete, extent.

In previous studies, another ATF was proposed that employed the same physical model but instead estimated the model parameters by exploiting the fact that central (ascending aortic) blood flow is negligible during diastole. It was also shown that this ATF could yield more accurate central BP estimates than GTFs when applied to femoral BP waveforms from animals. However, the systolic upstroke-downstroke intervals of the patient radial BP waveforms studied herein were often narrower than those of the femoral BP waveforms. As a result, the previous ATF sometimes predicted central blood flow waveforms with diastolic intervals that were too wide in this study. The conclusion is that the simple physical model of FIG. 2 may be more valid for the radial BP-to-central BP transfer function than the radial BP-to-central blood flow transfer function.

The ATF was assessed and compared to GTFs using the same patient data that helped popularize the GTF. These data included gold standard reference central BP waveforms in addition to non-invasive radial BP waveforms from 39 cardiac catheterization patients as well as some interventions to vary BP (see Table 1). The specific hypothesis was that changes in PP amplification (the ratio of radial PP to central PP) would adversely impact the GTFs but not the ATF. So, the patient data was divided into low, middle, and high PP amplification groups of equal sizes and studied the transfer function performance per group (see Table 2).

The GTF_{SphygmoCor}, which was able to mimic the SphygmoCor device, estimated central BP most accurately in the high, rather than middle, PP amplification group. The reason may be that the device was trained using central and radial BP waveforms from a large number of relatively healthy subjects but of similar average age as the patients studied herein. Hence, the performance of the GTF_{SphygmoCor} degraded with decreasing PP amplification and became relatively poor in the low PP amplification group. These results suggest that the SphygmoCor device may possibly be biased toward normal subjects.

The GTF_{ARX}, which was trained using the same data and in the same way as the ATF, accurately estimated central BP in the middle PP amplification group, as expected. Its performance degraded in the low PP amplification group but was surprisingly good in the high PP amplification group. Hence, although GTFs are population averages, they have some ability to adapt to variations in PP amplification by virtue of being frequency selective.

The ATF accurately estimated central BP in all three PP amplification groups. Further, its performance was significantly better than both GTFs. Most notably, in the low PP amplification group, the ATF was able to reduce the central TW, SP, and PP estimation errors by an average of nearly 50% compared to the GTF_{SphygmoCor} and 40% compared to the GTF_{ARX}. The low PP amplification group may not be an insignificant one. This group, by definition, constituted one-third of the patient data herein. Further, low PP amplification may occur with hypertension and aging and is caused by a short wave travel time to the radial artery and/or a small wave reflection coefficient.

The wave travel time (T_{d}) estimates of the ATF indeed decreased with decreasing PP amplification, while the wave reflection coefficient (Γ) estimates did not change. However, it is noted that the T_{d} estimates may be more reliable, because the transfer function is often relatively insensitive to Γ. In particular, the magnitude response of the transfer function is given as follows:

$$\sqrt{{cos}^{2} ( 2 π {T}_{d} f ) + {( \frac{1 - Γ}{1 + Γ} )}^{2} {sin}^{2} ( 2 π {T}_{d} f )}$$

where f is frequency. Hence, the transfer function is specifically insensitive to Γ for small f (e.g., <3 Hz, which is a crucial frequency band) and moderate to high Γ (e.g., >0.4) and becomes even more insensitive to Γ with decreasing T_{d}. Assuming Γ is relatively unimportant, if T_{d} is small, then the central BP waveform derived by the ATF will appear like the radial BP waveform, which nominally does not exhibit exponential diastolic decays. On the other hand, if T_{d} is large, then the derived central BP waveform will show double peaks rather than a smooth decay. Invoking the central BP exponential diastolic decay assumption may balance these two parameter settings so as to yield the proper T_{d} value. That is, if T_{d} were actually small (i.e., large pulse wave velocity), then the radial and central BP waveforms may both exhibit similar exponential diastolic decays, and the ATF would thus correctly yield a small T_{d} value. But, if T_{d} were actually large, then the radial BP waveform may not show an exponential diastolic decay, and the ATF would thus correctly yield a larger T_{d} value. In this way, the ATF was accurate over a wide range of PP amplifications.

An important issue left unaddressed in the validation studies is practical calibration of radial BP waveforms via applanation tometry. Like the original GTF validation studies, the radial BP waveforms were calibrated with the reference central BP waveforms in order to focus on the transfer function. However, a major source of error in non-invasive central BP estimates is calibration with error-prone brachial BP measurements via current oscillometric cuff devices. More accurate automatic cuff BP measurement methods are therefore also needed. Some have proposed a patient-specific method recently and combining it with the simple ATF introduced herein may achieve accurate, non-invasive central BP monitoring in practice. For example, reference may be made to U.S. Patent Application Publication No. 2014/0066793 and PCT Publication No. WO 2017/044823 which are incorporated herein in their entirety. This patient-specific method may also yield the entire brachial BP waveform. From this waveform, central BP may be estimated using the adaptive methods proposed herein. In this way, central BP may be computed only from a standard oscillometric arm cuff without requiring an additional pulse volume plethysmography measurement at fixed cuff pressure.

FIG. 6 depicts an exemplary system 60 that implements the techniques according to the present disclosure. The system 60 includes a model estimation module 62 that estimates parameters of the ATF and a diagnostic module 63 that identifies patient health conditions. The system 60 may further include a sensor 61 and one or more output devices, such as a display or a printer. However, it can be appreciated that the system 60 may include fewer or additional modules and/or sensors.

The sensor 61 measures a peripheral BP or related waveform from the subject. In one embodiment, the sensor 61 may measure the peripheral BP waveform invasively from the femoral or radial artery of the subject. For example, the sensor 61 may be a fluid-filled catheter. In other embodiments, the sensor 61 may measure the peripheral BP non-invasively. For example, the sensor may be a finger-cuff photoplethysmograph or an applanation tonometer or an oscillometric cuff. It is understood that other types of sensors and other measurement sites also fall within the scope of this disclosure.

The model estimation module 62 is configured to receive the measured peripheral BP waveform from the sensor. A model that relates the measured peripheral BP waveform to a central BP waveform is accessible to the model estimation module 62. In an example embodiment, the model is stored in a non-transitory data store (i.e., computer memory). Multiple sets of candidate values for wave travel time and wave reflection coefficient are also accessible to model estimation module 62. In one embodiment, the multiple sets of candidate values are preselected and stored in the non-transitory data store. In other embodiments, the sets of candidate values may be selected by a user or generated dynamically by a selection algorithm.

For each set of candidate values, the model estimation module 62 computes a candidate central BP waveform by applying the model with a given set of candidate values to the measured peripheral BP waveform. For each candidate central BP waveform, the model estimation module 62 fits an exponential to diastolic decay of a given candidate central BP waveform. Lastly, the model estimation module 62 determines central BP for the subject to be the candidate central BP waveform having smallest fitting error between the exponential and the diastolic decay.

The diagnostic module 63 analyzes the estimated central BP (and wave travel time and wave reflection coefficient) and determines a health condition of the subject and/or administers treatment to the subject based on the analysis of the central BP. The diagnostic module 63 receives the estimated central BP waveform from the model estimation module 62. In one embodiment, the diagnostic module 63 monitors cardiac output using the parameter estimates and the tube-load model. The diagnostic module 63 may also determine at least one parameter of the central BP waveform. For example, the at least one parameter may include systolic pressure, diastolic pressure, pulse pressure, and systolic ejection interval.

The diagnostic module 63 may also estimate a cardiovascular variable from the central BP waveform. For example, the diagnostic module 63 may estimate the cardiovascular variable from the estimated central BP waveform using a lumped parameter model. The cardiovascular variable may be further defined as one of proportional cardiac output, proportional stroke volume, proportional total peripheral resistance, proportional maximum left ventricular elastance, and absolute left ventricular ejection fraction. In one embodiment, the diagnostic module 63 may calibrate the proportional cardiovascular variable to an absolute value using one of a nomogram, a single absolute measurement of cardiac output (e.g., thermodilution), and a single absolute measurement of ventricular volume (e.g., echocardiography). In one embodiment, an alarm is triggered upon excessive changes in any of the estimated variables. Lastly, the diagnostic module 63 may administer therapy to the subject, or modify the subject's therapy, based on one or more cardiovascular variables obtained according to the various methods presented herein.

The display 64 is configured to receive and display any of the derived waveforms and/or parameters noted above. For example, doctors and/or nurses may observe the estimated central BP waveform to diagnose a condition of the subject or to monitor a condition of the subject. However, it can be appreciated that other types of output devices may be used in lieu of the display device.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for determining central blood pressure for a subject, comprising:
measuring, by a sensor, a peripheral blood pressure waveform from the subject;
defining a model that relates the measured peripheral blood pressure waveform to a central blood pressure waveform, where the model is defined in terms of parameters representing wave travel time and wave reflection coefficient;
determining central blood pressure for the subject by selecting the parameters and applying the model to the measured peripheral blood pressure in a manner that yields smallest error in fitting of an exponential function to a diastolic interval of the central blood pressure.

2. The method of claim 1 further comprises measuring the peripheral blood pressure waveform from a radial artery of the subject.

3. The method of claim 1 further comprises measuring the peripheral blood pressure waveform using a catheter or a finger-cuff photoplethysmograph or an applanation tonometer or an oscillometric cuff.

3. The method of claim 1 wherein the model is a tube-load model, where the tube represents wave travel path between central aorta and a peripheral artery and terminal loads represent the arterial bed distal to the peripheral artery.

4. The method of claim 1 wherein the model is defined as
$${P}_{c} ( t ) = \frac{1}{1 + Γ} {P}_{r} ( t + {T}_{d} ) + \frac{Γ}{1 + Γ} {P}_{r} ( t - {T}_{d} )$$
where P_{c}(t) is the central blood pressure waveform, Pᵣ(t) is the measured peripheral blood pressure waveform, T_{d} is the wave travel time and Γ is the wave reflection coefficient.

5. The method of claim 1 wherein determining central blood pressure further comprises
selecting multiple sets of candidate values for the wave travel time and the wave reflection coefficient from respective physiological ranges of values;
computing, for each set of candidate values, a candidate central blood pressure waveform by applying the model with a given set of candidate values to the measured peripheral blood pressure waveform;
fitting, for each candidate central blood pressure waveform, an exponential to the diastolic decay of a given candidate central blood pressure waveform; and
determining central blood pressure as the candidate central blood pressure waveform having smallest fitting error between the exponential and the diastolic decay.

6. The method of claim 5 further comprises low pass filtering each candidate central blood pressure waveform prior to the step of fitting.

7. A method for determining central blood pressure for a subject, comprising:
measuring, by a sensor, a peripheral blood pressure waveform from the subject;
defining a model that relates the measured peripheral blood pressure waveform to a central blood pressure waveform, where the model is defined in terms of parameters representing wave travel time and wave reflection coefficient;
selecting multiple sets of candidate values for the model parameters;
computing, for each set of candidate values, a candidate central blood pressure waveform by applying the model with a given set of candidate values to the measured peripheral blood pressure waveform;
fitting, for each candidate central blood pressure waveform, an exponential to the diastolic decay of a given candidate central blood pressure waveform; and
determining central blood pressure as being the candidate central blood pressure waveform having smallest fitting error between the exponential and the diastolic decay, where the steps of computing, fitting and determining are executed by a computer processor of a computing device.

8. The method of claim 7 further comprises measuring the peripheral blood pressure waveform using a catheter or a finger-cuff photoplethysmograph or an applanation tonometer or an oscillometric cuff.

9. The method of claim 7 wherein the model is a tube-load model, where the tube represents wave travel path between central aorta and a peripheral artery and terminal loads represent the arterial bed distal to the peripheral artery.

10. The method of claim 7 wherein the model is defined as
$${P}_{c} ( t ) = \frac{1}{1 + Γ} {P}_{r} ( t + {T}_{d} ) + \frac{Γ}{1 + Γ} {P}_{r} ( t - {T}_{d} )$$
where P_{c}(t) is the central blood pressure waveform, Pᵣ(t) is the measured peripheral blood pressure waveform, T_{d} is the wave travel time and Γ is the wave reflection coefficient.

11. The method of claim 7 further comprises selecting multiple sets of candidate values for wave travel time and wave reflection coefficient from respective physiological ranges of values.

12. The method of claim 7 further comprises low pass filtering each candidate central blood pressure waveform prior to the step of fitting.

13. The method of claim 7 wherein fitting an exponential further comprises
estimating a diastolic interval of the given candidate central blood pressure waveform using pulse length; and
applying a logarithm operation to the estimated diastolic interval of the given candidate central blood pressure waveform and fitting a line to the log transformed data.

14. A method for determining central blood pressure for a subject, comprising;
measuring, by a sensor, a peripheral blood pressure waveform from the subject;
defining a model that relates the measured peripheral blood pressure waveform to a central blood pressure waveform, where the model is defined in terms of parameters representing the wave travel time and wave reflection coefficient;
determining the parameters representing the wave reflection coefficient based on population averages;
determining the parameters representing wave travel time based on its inverse relationship with blood pressure; and
determining central blood pressure by applying the determined model to the measured peripheral blood pressure waveform, where the step of determining is executed by a computer processor of a computing device.

15. The method of claim 14 further comprising measuring the peripheral blood pressure waveform using an oscillometric cuff.

16. The method of claim 15 wherein the cuff is set to a constant pressure and the resulting pulse volume plethysmography waveform is calibrated to the blood pressure levels determined by inflation and deflation of the cuff.

17. The method of claim 14 wherein the wave travel time is determined by a regression equation involving a level of the measured peripheral blood pressure waveform.

18. A computer-implemented system for determining central blood pressure for a subject, comprising:
a sensor configured to measure a peripheral blood pressure waveform of the subject;
a data store that stores a model that relates the measured peripheral blood pressure waveform to a central blood pressure waveform, where the model is defined in terms of wave travel time and wave reflection coefficient;
a model estimation module configured to receive the measured peripheral blood pressure waveform from the sensor and to receive multiple sets of candidate values for wave travel time and wave reflection coefficient,
wherein the model estimation module computes, for each set of candidate values, a candidate central blood pressure waveform by applying the model with a given set of candidate values to the measured peripheral blood pressure waveform and fits, for each candidate central blood pressure waveform, an exponential to diastolic decay of a given candidate central blood pressure waveform, wherein the model estimation module is computer readable instructions executed by a computer processor residing on a computing device.

15. The system of claim 18 wherein the model estimation module determines central blood pressure for the subject to be the candidate central blood pressure waveform having smallest fitting error between the exponential and the diastolic decay.

19. The system of claim 18 wherein the sensor is a finger-cuff photoplethysmograph or an applanation tonometer or a catheter or an oscillometric cuff.

20. The system of claim 18 wherein the model is a tube-load model, where the tube represents wave travel path between central aorta and a peripheral artery and terminal loads represent the arterial bed distal to the peripheral artery.

21. The system of claim 18 wherein the model is defined as
$${P}_{c} ( t ) = \frac{1}{1 + Γ} {P}_{r} ( t + {T}_{d} ) + \frac{Γ}{1 + Γ} {P}_{r} ( t - {T}_{d} )$$
where P_{c}(t) is the central blood pressure waveform, Pᵣ(t) is the measured peripheral blood pressure waveform, T_{d} is the wave travel time and Γ is the wave reflection coefficient.
